(19) United States
(12) Patent Application Publication
(10) Pub. No.: US 2016/0243271 A1
(43) Pub. Date: Aug. 25, 2016

(54) Essential Oils Or Volatile Organics Thereof Electrospun In Chitosan Nanofiber Mats

(71) Applicant: UNIVERSITY OF MASSACHUSETTS, Boston, MA (US)

(72) Inventors: Jessica D. Schiffman, Amherst, MA (US); Katrina A. Rieger, Amherst, MA (US)

(21) Appl. No.: 15/027,359

(22) PCT Filed: Oct. 8, 2014
(86) PCT No.: PCT/US14/59678

§ 371 Date: Apr. 5, 2016

Related U.S. Application Data
(60) Provisional application No. 61/888,164, filed on Oct. 8, 2013.

(51) Int. Cl. A61L 15/20; A61L 15/42; A61L 15/28; A61L 15/44; A61L 15/22; A61L 15/26

(52) U.S. Cl. CPC A61L 15/20; A61L 15/225; A61L 15/26; A61L 15/28; A61L 15/44; A61L 15/425; A61L 2300/404; D01D 5/003

(57) ABSTRACT

Nanofiber mats comprising chitosan, chitosan-essential oils and, in particular, chitosan-cinnamaldehyde (chitosan-CA) are described, including methods of making the nanofiber mats and methods of using same, for example, for inactivating bacteria such as E. coli and P. aeruginosa, and as therapeutic wound dressings to treat nosocomial pseudomonas infections.

### RELATED APPLICATION(S)

This application claims the benefit of U.S. Provisional Application No. 61/888,164, filed on Oct. 8, 2013. The entire teachings of the above application are incorporated herein by reference.

### BACKGROUND OF THE INVENTION

Ubiquitously, planktonic bacteria attach to surfaces and develop into communities of microorganisms called biofilms.¹ Delaying the onset of biofilm formation is imperative due to the high mortality rate that they impose to both immunocompromised and critically-ill patients. Hospitalized patients who are critically ill have a 40-60% likelihood of dying from an infection caused by the leading Gram-negative opportunistic human pathogen, Pseudomonas aeruginosa (P. aeruginosa).^{2,3} In U.S. hospitals alone, 1.7 million patients a year are inflicted with healthcare associated infections with nearly 99,000 cases resulting in death.⁴

The severity of nosocomial pseudomonas infections is augmented by the increasing tolerance of this microorganism to current antipseudomonal drugs, such as piperacillin, ceftazidime, imipenem, and ciprofloxacin.^{5,6} The low permeability of its outer membrane provides P. aeruginosa with an intrinsic resistance to antibiotics. When free-floating, the microorganisms attach to surfaces and form 3-dimensional structures called biofilms, within which P. aeruginosa becomes 1000 times more resistant to antibiotics than when the bacteria were in the planktonic state.^{7,8} The stages of biofilm development include the (i) initial reversible attachment, (ii) irreversible attachment, (iii) early maturation, (iv) maturation, and (v) dispersion of the bacteria. Growth and maturation of P. aeruginosa biofilms are coordinated by a cell-to-cell communication mechanism know as quorum sensing (QS).⁸ In P. aeruginosa, two QS systems are activated upon biofilm formation: lasB during irreversible attachment and rhlA during the early maturation stage.⁹ Thus, developing new strategies that can delay the onset of biofilm formation, while avoiding the spread of resistance genes are in high demand.

For centuries, plant essential oils have been used to fight bacterial infections due to their broad-range effectiveness against a wide assortment of bacteria.¹⁰ The antimicrobial mechanisms of many essential oils have been investigated and are the subject of numerous review articles.¹¹⁻¹³ The hydrophobicity of the small terpenoid and phenolic compounds is related to their complex antimicrobial mechanism—they can easily permeate the cell membrane leading to a depletion of the proton gradient and subsequent disruption of ATP synthesis or cell lysis.¹² Since their exact antimicrobial mechanism is complex, the development of resistance by bacteria is difficult. A well-studied essential oil is cinnamon oil, whose major component is cinnamaldehyde (CA). CA decreases metabolic activity and the replication rate of P. aeruginosa.¹⁵ In P. aeruginosa, exposure to CA leads to a coagulation of cytoplasmic material and an extrusion of intracellular content. CA inhibits swimming motility in Escherichia coli (E. coli)¹⁶ and sublethal concentrations of CA act as a QS inhibitor, interfering with the autoinducer-2 (AI-2) system.^{7,17,18} Therefore, the use of CA at low concentrations could prevent colonization of bacteria without exerting evolutionary pressure. Previously, Zodrow et al.¹⁹ encapsulated CA and carvacrol (from oregano) within solid poly(lactic-co-glycolic acid) (PLGA) films. However, these films lack the porosity and permeability required for wounds to heal properly.²⁰ Accordingly, there is a need for improved products that can incorporate and release essential oils. Additionally, there is a need for such products that avoid the use of potentially toxic surfactants.

### SUMMARY OF THE INVENTION

A method of preparing a nanofiber mat having an essential oil or a volatile organic thereof includes electrospinning a mixture of chitosan, poly(ethylene oxide), an acid, and an essential oil, wherein the mixture of has a pH of approximately 4, to thereby prepare a nanofiber mat having an essential oil. The ratio of chitosan to poly(ethylene oxide) can be approximately 1:1. The method can further include mixing the chitosan and poly(ethylene oxide) for approximately 24 hours. The acid can be acetic acid. The essential oil can be cinnamaldehyde (CA). The molecular weight of the chitosan is between about 460,000 Da and about 1,000,000 Da. The amount of cinnamaldehyde can be approximately 0.5 v/v % or approximately 5.0 v/v %. The separation distance between the needle and collection plate can be approximately 120 mm to approximately 160 mm. The applied voltage for the electrospinning can be approximately 25 kV. The individual nanofibers within the nanofiber mat can have a diameter of between approximately 38 nm and 55 nm.

A method of inactivating microbes (microscopic organisms that cause disease, contamination, or infection including bacteria, viruses, fungi, and protozoa) can include contacting a nanofiber mat as disclosed herein with the microbe under time and conditions sufficient for the microbe to be partially, or completely, inactivated. The microbe can be bacteria, such as E. coli or P. aeruginosa. The microbe can have formed a biofilm and the time and conditions the nanofiber mat is contacted with a microbial biofilm can be sufficient for the dispersion of the biofilm.

### BRIEF DESCRIPTION OF THE DRAWINGS

FIG. 1 is a proposed mechanism for the reaction of chitosan with cinnamaldehyde.

FIG. 2 is the ¹H NMR spectra of the chitosan derivatives synthesized with (top to bottom) 0%, 0.5%, and 5.0% CA.

FIGS. 3A, 3C, and 3E are scanning electron micrographs (SEM) displaying the morphology of chitosan/CA/PEO nanofiber mats containing (A) 0%, (C) 0.5%, and (E) 5.0% CA. FIGS. 3B, 3D, and 3F are fiber diameter distribution charts for chitosan-CA nanofiber mats containing (B) 0%, (D) 0.5%, and (F) 5.0% CA as determined using ImageJ software.

FIG. 4 is the solid state ¹³C NMR spectra of (top-to-bottom) chitosan:PEO powder (control) and electrospun chitosan/CA(0, 0.5, and 5.0%) nanofiber mats. Peaks between 120 and 150, as well as at 170 ppm confirm the presence of CA in the chitosan/CA(0.5 and 5.0%)/PEO nanofiber mats.

FIG. 5A displays the quantity of CA liquid released from chitosan-CA(0.5% and 5.0%) nanofiber mats as determined through UV-visible spectroscopy at an absorbance of 293 nm. FIG. 5B displays the amount of CA vapor released from the chitosan-CA(0.5% and 5.0%) nanofiber mats as determined by gas chromatography. Release experiments were conducted using 10 mm×20 mm nanofiber mats submerged in (A) 1.5 mL and (B) 10 mL of an isotonic solution (pH 5.7). Error bars indicate one standard deviation.

FIG. 6 shows the loss of E. coli viability as a function of incubation time for chitosan-CA(0%, 0.5%, and 5.0%) nanofiber mats. Experiment were performed in triplicate and error bars indicate one standard error.

FIG. 7 shows the loss of P. aeruginosa viability as a function of incubation time for chitosan-CA(0%, 0.5%, and 5.0%) nanofiber mats. Asterisks (**) denote that a statistical increase in cytotoxicty occurred whereas “NS” indicate that the change is not significant. Experiment performed in triplicate and error bars indicate one standard error.

FIG. 8 displays the maximum incorporation of CA and H-CA into LMW CS, MMW CS, and MOD-MMW CS.

FIGS. 9A-C shows the rheological characterization of LMW CS, MMW CS, and MOD-MMW CS solutions containing no oil, CA, and H-CA.

FIG. 10 shows the fiber morphology (droplets, bead-on-string, or continuous fibers) produced from electrospinning LMW CS, MMW CS, and MOD-MMW CS with CA and H-CA.

### DETAILED DESCRIPTION OF THE INVENTION

Electrospinning provides an inexpensive and scalable process to generate flexible and highly porous nanofiber mats appropriate for biomedical applications.²¹ The electrospinning process forces a charged precursor solution out of a capillary, which is connected to a grounded collector. As the voltage is increased, the repulsive electrical forces pull the pendent drop into a Taylor Cone. At a critical voltage, the electrical forces overcome the surface tension forces resulting in the emergence of a liquid jet. As the jet is stretched and whipped solvent instantly begins to evaporate and solid fibers are formed.

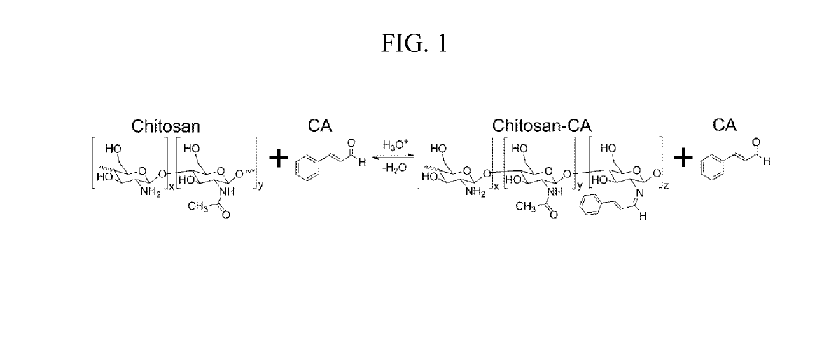

Chitosan, a copolymer of 2-acetamido-2-deoxy-D-glucopyranose and 2-amino-2-deoxy-D-glucopyranose units joined by β(1→4) bonds, was chosen as one of the two polymer constituents of the nanofiber mats. Due to the availability of primary amine groups on chitosan, CA can be attached using a Schiff base reaction (FIG. 1), which is enhanced at pHs between 3 and 5.^{22,23} Additionally, chitosan exhibits inherent antimicrobial activity. Without wishing to be bound by theory, it is believed that the positive charge on the C-2 of the glucosamine monomer interacts with the negative charge on the microbial cell membrane to provide the antimicrobial activity.²⁴ A second polymer constituent, poly(ethylene oxide) (PEO), was utilized in order to use aqueous acetic acid as the solvent and maintain an appropriate solution pH of 4. The pH of 4 is appropriate because here the Schiff base reaction was enhanced.²⁵

As explained in detail herein, chitosan and in-house synthesized chitosan-CA derivatives were electrospun along with PEO into nanofiber mats. CA release studies at physiological conditions were correlated to time-dependent bacterial cytotoxicity studies. The chitosan and chitosan-CA derivative nanofiber mats exhibited high levels of antibacterial efficacy against two Gram-negative bacteria, E. coli and P. aeruginosa. Chitosan-derivative nanofibers mats that release CA can serve as therapeutic wound dressings to treat nosocomial pseudomonas infections.

Chitosan nanofiber mats can also be synthesized with a wide variety of compounds that can react with the amine group of chitosan. In particular, compounds that react to form an imine with amine other essential oils, small molecules, and fragrances. For example, chitosan nanofiber mats can be synthesized with essential oils, such as vanillin, citronellal, lemongrass, and thymol. Chitosan nanofiber mats can also be synthesized with fragrances, such as lilac and lyral. Chitosan nanofiber mats can also be synthesized with sugars or carbohydrates, such as glucose and lactose. Chitosan nanofiber mats can also be synthesized with vitamins, such as vitamin B6 analogues. One of ordinary skill in the art would expect that different compounds would exhibit different release kinetics, and would adjust the amounts of the compounds accordingly. Chitosan nanofiber mats synthesized with the compounds disclosed herein have a number of applications, including use as an antibacterial, an anticorrosive, or in wound healing. The mats can be used as surface coatings or as wound dressings.

### EXEMPLIFICATION

### Example 1

### Materials and Methods

Materials and Chemicals.

All compounds were used as received. Low molecular weight chitosan (M_{w}=460,000 Da), poly(ethylene oxide) (PEO, M_{w}=600,000 Da), cinnamaldehyde (CA, ≧93%, FG, M_{w}=132.16 Da), analytical reagent grade acetic acid (AA), sodium chloride (NaCl), glutaraldehyde (GA, 50 wt % aqueous solution), deuterium oxide, and acetic acid-d₄ (AA-d₄) were obtained from Sigma-Aldrich (St. Louis, Mo.). Sodium hydroxide (NaOH) was obtained from Fisher Scientific (Fair Lawn, N.J.). Difco Luria-Bertani (LB) broth was purchased from BD Life Sciences (Franklin Lakes, N.J.). Propidium iodide (PI) and 4′,6-diamidino-2-phenylindole (DAPI) were purchased from Invitrogen (Carlsbad, Calif.). Deionized (DI) water was obtained from a Barnstead Nanopure Infinity water purification system (Thermo Fisher Scientific, Waltham, Wash.).

Chitosan/CA Schiff Base Quantification.

Proton nuclear magnetic resonance (¹H NMR, Bruker Avance 400) along with SpinWorks3, an NMR analysis software, were employed to quantitatively determine the degree of acetylation (DA) and degree of substitution (DS) of the chitosan. Chitosan (2.5w/v %, 15 mg) was dissolved in AA-d4 (600 μL) using an Arma-Rotator A-1 (Bethesda, Mass.) to determine the DA. CA was added (0, 0.5, and 5.0 v/v % CA) to 2.5 w/v % chitosan dissolved in 0.5 M AA-d₄ (600 μL) to determine the DS. DA values were determined by taking the relative integrals of 1.7-2.4 ppm over 2.7-4.4 ppm.

Chitosan/CA/PEO Nanofiber Mat Fabrication.

A 1:1 ratio of chitosan to PEO (0.5 g:0.5 g) in 0.5 M AA (20 mL) corresponding to a 5.0 w/v % solution was mixed for 24 h at 20 rpm using an Arma-Rotator A-1 (Bethesda, Mass.). Various amounts of CA(0 mL, 0.1 mL, and 1.0 mL corresponding to 0, 0.5, and 5.0 v/v %, respectively) were added to the solution and mixed for an additional 24 h at which point, the solution changed from transparent to white. Throughout the mixing process, the solution had a pH value of 4.

Each chitosan-CA:PEO solution was loaded into a 5 mL Luer-Lock tip syringe capped with a Precision Glide 18 gauge needle (Becton, Dickinson & Co. Franklin Lakes, N.J.), which was secured to a PHD Ultra syringe pump (Harvard Apparatus, Plymouth Meeting, Pa.). Alligator clips were used to connect the positive anode of a high-voltage supply (Gamma High Voltage Research Inc., Ormond Beach, Fla.) to the needle and the negative anode to a copper plate wrapped in aluminum foil. A constant feed rate of 60 μL/min, an applied voltage of 25 kV, and a separation distance of 120, 160, and 140 mm were used to electrospin chitosan/CA(0, 0.5, and 5.0%)/PEO solutions. The assembled electrospinning apparatus was housed in an environmental chamber (CleaTech, Santa Ana, Calif.) with a desiccant unit (Drierite, Xenia, Ohio) to maintain a temperature of 22±1° C. and a relative humidity of 24-28%. All nanofiber mats used in release and bacterial studies were spun for 1 hr. For simplicity, instead of being called chitosan/CA(0, 0.5, or 5.0% CA)/PEO nanofiber mats, they will be referred to as 0, 0.5, and 5.0% chitosan-CA nanofiber mats. As-spun chitosan-CA mats were crosslinked using GA vapor as previously described.²⁵ Briefly, mats were placed in a vapor chamber (122 mm×98 mm×78 mm, Biohit Inc, Neptune, N.J.) containing 1.0 mL of GA liquid, 50 wt % aqueous solution. At room temperature (23° C.), the GA liquid vaporized and was allowed to crosslink the fibers for 4 hr. This ensured the chemical stability of the nanofiber mats for release studies and antibacterial evaluation.

Chitosan/CA/PEO Nanofiber Mat Characterization.

Micrographs were acquired using a FEI-Magellan 400 scanning electron microscope (SEM). A Gatan high resolution ion beam coater model 681 was used to sputter coat samples for 4 min with platinum. Fiber diameter distribution was determined by Image J 1.45 software (National Institutes of Health, Bethesda, Md.) by measuring 50 random fibers from 5 micrographs. Carbon-13 NMR (¹³C NMR, DSX300) and SpinWorks3 were employed to confirm the presence of chitosan, PEO, and CA within the as-spun mats. Approximately 50 mg of chitosan/CA(0, 0.5, and 5.0%)/PEO nanofiber mat, as well as a 100 mg powder sample of chitosan:PEO (1:1) were analyzed.

Liquid and Vapor State Release of CA from Chitosan/CA/PEO Nanofiber Mat.

The release characteristics of CA as a liquid (CA-liquid) and as a vapor (CA-vapor) from the nanofiber mats were quantified at times relevant to antibacterial activity experiments. Consistent chitosan/CA(0, 0.5, and 5.0%)/PEO nanofiber mats (10 mm×20 mm) were placed into 1.5 mL centrifuge tubes containing 1.0 mL of an isotonic solution (0.9% NaCl, pH 5.7) with virtually no headspace. The samples were maintained at 37° C. and shaken at 100 rpm. As a function of time, all of the isotonic solution (1.0 mL) was removed via needle/syringe and placed in a cuvette for analysis. After testing, the 1.0 mL was returned to the vial until the last time point. The amount of CA-liquid present in the isotonic solution was determined using UV-Visible spectroscopy (UV-Vis, Agilent Diode Array) at an absorbance of 293 nm, as previously reported for CA.^{27,28} The solution was placed back into the sample to maintain the same gradient flux.

As a function of time, quantification of the CA-vapor released from the mat into the headspace of the vial was performed using gas chromatography, GC (Agilent/HP 6890 equipped with Flame Ionization Detector FID).²⁹ Nanofiber mats (10 mm×20 mm) were placed in 10 mL of isotonic solution with 10 mL of headspace in the vial for the CA-vapor to accumulate. The sealed vials were kept at 37° C. and shaken at 100 rpm. Manual samples containing 1 mL of gas were injected into the GC at 30, 90, and 180 min intervals. Standard calibration curves were determined for both UV-Vis and GC to quantify the amount of CA in each sample. Additionally, chitosan-CA (0%) mats were used as controls.

Evaluation of Antibacterial Activity of Electrospun Chitosan/CA/PEO Nanofiber Mats.

The model organisms were Escherichia coli S20918 (E. coli) and P. aeruginosa S20930 (P. aeruginosa), purchased from Thermo Fisher Scientific, Waltham, Mass. The bacteria were grown in LB at 37.0 and harvested at a concentration of 108 cells/mL. To remove residual macromolecules and other growth medium constituents, cells were washed twice and then resuspended in an isotonic solution (0.9% NaCl, pH 5.7). This isotonic solution was used for E. coli and P. aeruginosa loss of viability studies.

The viability loss of E. coli in contact with chitosan/CA(0, 0.5, and 5.0%)/PEO nanofiber mats was assayed according to a modified ASTM E2149-01 shake flask method.³⁰ In parallel to each experiment (i) a blank control (no nanofiber mat) and (ii) an MIC control of CA for E. coli (3 mM CA in solution) were employed.¹⁹ Electrospun mats (10 mm×20 mm) were placed into culture tubes (16 mm×125 mm) along with 4 mL suspensions of E. coli or P. aeruginosa (5×10⁶ cells/mL) in an isotonic solution (0.9% NaCl, pH 5.7). The suspensions were shaken at 200 rpm in an orbital shaker maintained at 37° C. At 30, 90, and 180 minutes, 100 μL were pipetted out from the tubes and serial dilutions were plated onto LB plates using the spread plate method.³⁰⁻³² After 24 hr incubation on agar plates, the number of viable bacteria was counted. The number of viable cells was multiplied by the dilution factor and expressed as the mean colony forming unit (CFU) per mL. The percent reduction of bacteria resulting from contact with the nanofiber mats was determined using Equation 1 where A represents the mean log₁₀ density of bacteria for the flask containing the treated substrate after the specified contact time and B represents the untreated substrate after the specified contact time.

$$Reduction of Bacteria ( \% ) = \frac{B - A}{B} \times 100 \qquad ( 1 )$$

Viability loss of P. aeruginosa in contact with chitosan/CA(0, 0.5, and 5.0%)/PEO nanofiber mats was determined using a previously described fluorescence assay.³³ Electrospun mats (20 mm×10 mm) were placed at the bottom of 35 mm×10 mm petri dishes (Becton, Dickinson & Co., Franklin Lakes, N.J.) to which the cells (10⁷ cells/mL) resuspended in an isotonic solution were added and diluted by a factor of 2. The cells were incubated at 37° C. for various times (30, 90, and 180 min). At each time point, cells were stained in the dark with PI (excitation/emission at 535 nm/617 nm) for 15 min and then counter-stained with DAPI (excitation/emission at 358 nm/461 nm). Fluorescence images were acquired to detect the cells utilizing an epifluorescence microscope (Olympus) with a Chroma cube filter. Five representative images were taken at 20× magnification at various locations for each specimen. Dead cells and the total number of cells were determined by direct cell counting. Loss of viability was taken as the ratio of the number of cells stained with PI divided by the number of cells stained with DAPI plus PI. Throughout the Results and Discussion section, all statistical differences were determined using an unpaired t-test with values of p≦0.05 considered to be statistically significant.

### Example 1

### Results and Discussion

Chitosan-CA Derivative Characteristics.

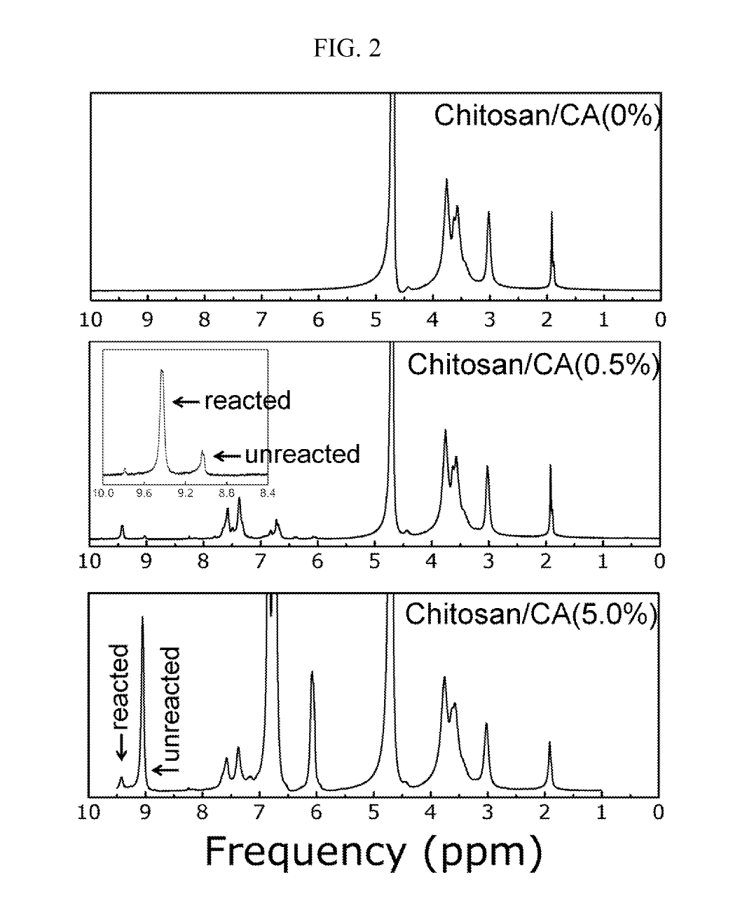

To better quantify the attachment efficiency of cinnamaldehyde (CA) to chitosan, the structure of the copolymer was first characterized. The degree of N-acetylation (DA) for chitosan is defined as the fraction of N-acetylated glucosamine units (FIG. 1, y-block) to the total glucosamine units (FIG. 1, x and y-block).^{34,35} Chitosan is dominated by glucosamine residues whose free amino groups at the C-2 position make the biopolymer soluble in weakly acidic solutions. Additionally, a reversible Schiff base reaction can also occur on the free amines. The DA of the chitosan was determined to be 13% via proton nuclear magnetic resonance spectroscopy (¹H NMR, FIG. 2, top) using the detailed procedure outlined by Femandez-Megia et al.³⁶ Briefly, DA values were determined by taking the relative integrals of 1.7-2.4 ppm over 2.7-4.4 ppm.³⁴⁻³⁶

The degree of substitution (DS), the ratio of amines reacted with CA over the total amount of amines³⁷, was characterized using the ¹H NMR peaks at 9.0 ppm and 9.5 ppm³⁸, which correspond to the presence of unreacted and reacted CA, respectively (FIG. 2). DS values were calculated from the ratio of the integrated resonances of reacted CA (9.25-9.6 ppm) over glucosamine residues on chitosan (2.7-4.4 ppm). It was determined that chitosan solutions mixed with 0.5 and 5.0% CA yielded comparable DS values, 15% and 16%, respectively. While our initial chitosan had 87% free amines, after mixing the chitosan/CA(0.5 and 5.0%) derivatives had approximately 70% free amines remaining Importantly, the ¹H NMR spectra revealed that unreacted CA is also present indicating that chitosan is physically entrapping the volatile oil. The chitosan/CA(0.5%) derivative contains 28% unreacted CA, while the chitosan/CA(5.0%) derivative contains a statistically higher, 89% unreacted CA.

Chitosan-CA Nanofiber Mat Characteristics.

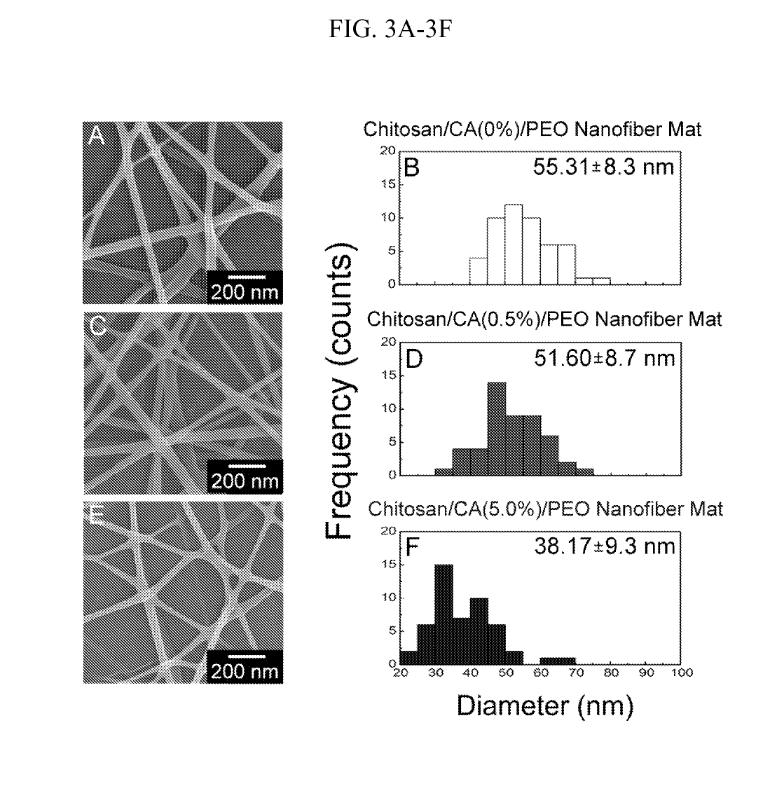

Chitosan/CA(0%)/PEO nanofiber mats were successfully electro-spun from a 5.0 w/v % solution containing an equal mass ratio of the two polymers. Maintaining a solution pH of 4 (i) facilitates chitosan-CA derivative synthesis and (ii) retains attachment until the mats are exposed to a physiologically relevant pH. To keep an appropriate solution pH of 4, chitosan was electrospun along with poly(ethylene oxide) (PEO), a biocompatible and biodegradable polymer.⁴⁰ As displayed on FIG. 3A, the chitosan/CA(0%)/PEO nanofiber mats were composed of continuous, fine, cylindrical fibers, consistent with the morphology previously reported.^{26,50} Nanofiber mats were also effectively electrospun from solutions containing the two different CA loadings, chitosan/CA(0.5 and 5.0%)/PEO with the polymers at a 1:1 ratio (FIGS. 3C and E).

Average fiber diameters (n=50) for the electrospun chitosan/CA(0, 0.5, and 5.0%)/PEO nanofiber mats are displayed in FIG. 3. The chitosan/CA(0, 0.5, and 5.0%)/PEO nanofiber mats had average fiber diameters of 55±8 nm, 52±9 nm, and 38±9 nm, respectively, which was similar to the chitosan/PEO nanofiber mats previously spun in literature. An unpaired t-test between the chitosan/CA(0%)/PEO and the chitosan/CA(0.5 and 5.0%)/PEO mats determined that the variation in fiber diameter was not statistically significant. Simply put, on average, by slight modification to our electrospinning parameters, we were able to produce fiber diameters that did not change due to the presence of CA.

Attempts to electrospin solutions containing only PEO and CA (no chitosan) phase separated: a homogeneous solution appropriate for electrospinning could not be obtained. Without wishing to be bound by theory, this suggests that in our system the chitosan is (i) chemically reacting with CA as observed in ¹H NMR and (ii) acts as a stabilizer to physically incorporate the CA into the precurser solution.

In order for release studies and antibacterial testing to be conducted, increased chemical stability of chitosan/CA(0, 0.5, and 5.0%) nanofiber mats was achieved using a slightly modified glutaraldehyde crosslinking protocol.²⁵ The carbonyl groups of glutaraldehyde can form a Schiff base reaction with the free amino groups at the C-2 position of chitosan and an acetalization reaction with the hydroxyl groups at the C-6 position of chitosan. Based on our ¹H NMR results, all chitosan/CA(0, 0.5, 5.0%) derivative solutions (0, 0.5, and 5.0%) have an abundant quantity of free amine groups ranging from 84-71% for both crosslinking and antibacterial activity. After crosslinking for 4 h, the average fiber diameter for the crosslinked chitosan/CA(0, 0.5, and 5.0%)/PEO fiber mats were statistically the same as the as-spun chitosan/CA(0, 0.5, and 5.0%)/PEO mats: 53.3±8 nm for 0% CA, 56.3±11 nm for 0.5%, and 41.1±6 nm for 5.0% CA. SEM micrographs of the crosslinked nanofiber mats 72 h post-submersion in acidic, neutral, and basic solutions (not shown) confirmed that the nanofibrous morphology of the mats was retained. Additionally, after the release studies and antibacterial evaluation described below, acquired SEM micrographs confirmed that the nanofiber morphology remained intact (not shown).

Chitosan/CA/PEO Nanofiber Mat Chemical Characteristics.

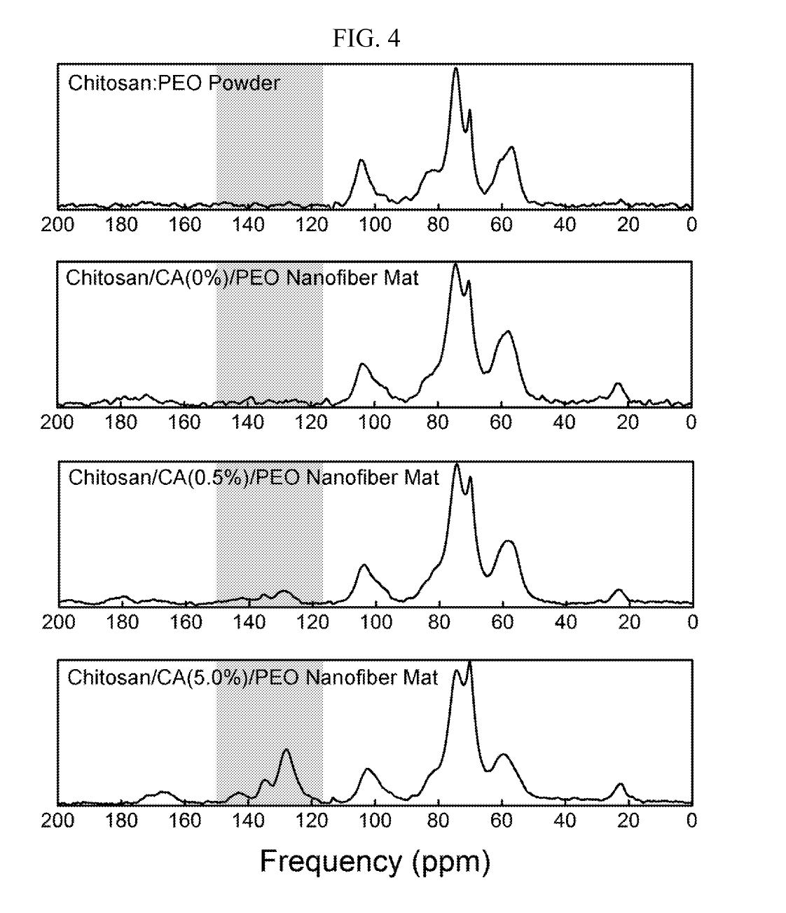

To qualitatively ensure that attached and unattached CA were present in the as-spun nanofiber mats, solid state ¹³C NMR was conducted (FIG. 4). The spectra suggests that due to the presence of CA (i) the height of the aromatic peak is greater for nanofibers spun with 5.0% CA than those spun from 0.5% CA, while (ii) the imine peak at 170 ppm is smaller thanthat observed for the aromatic peaks.⁵¹ This corroborates the solution NMR data: the majority of CA within the nanofiber mat is physically entrapped, unreacted CA. These peaks are absent from the control spectra acquired on chitosan/CA(0%)/PEO nanofiber mats and chitosan:PEO powder. “Control” chitosan/CA/PEO nanofiber mats electrospun from the same solvent system with only unreacted CA could not be obtained as the Schiff base is pH dependent. Further increasing the pH (beyond where Schiff bases are encouraged) resulted in chitosan that was not fully dissolved.

Release Characteristics of Chitosan-CA Nanofiber Mats.

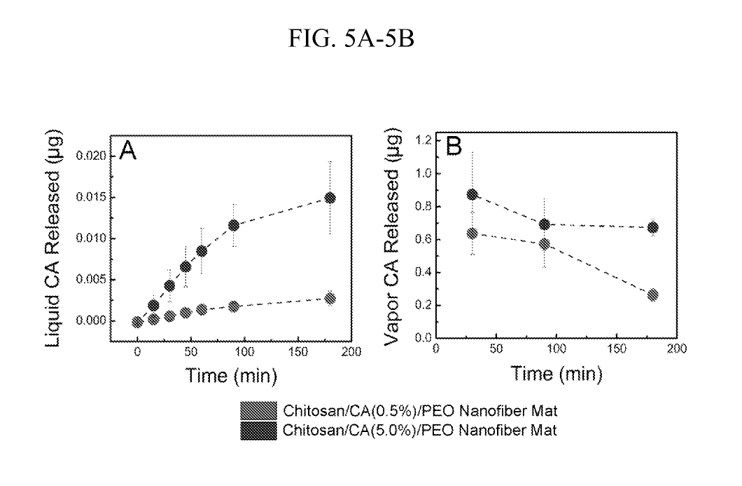

Using UV-Visible spectroscopy (UV-Vis, FIG. 4A) and gas chromatography (GC, FIG. 4B) the release characteristics of CA-liquid and CA-vapor from the nanofiber mats were quantified at times relevant to antibacterial activity tests at physiological temperature (37° C.) and pH (5.7). Preliminary experiments determined that using small, 1.5 mL vials, enabled the acquisition of the most accurate UV-Vis data because the CA-liquid remained highly concentrated. FIG. 5A displays that as a function of time, the release of CA continues to gradually increase from the chitosan/CA(0.5% and 5.0%)/PEO nanofiber mats. After 15 min, the chitosan/CA(0.5% and 5.0%)/PEO nanofiber mats demonstrated an initial CA-liquid release of 2.0×10⁻³±3×10⁻⁴ μg and 1.9×10⁻³±1×10⁻⁴ μg. At longer times (90 and 180 min), the amount of CA release reached started to level off indicating that equilibrium has been reached. Specifically, CA-liquid released reached quantities of 2.7×10⁻³±9×10⁻⁴ μg and 1.5×10⁻²±4×10⁻³ μg after 180 min for the chitosan/CA(0.5% and 5.0%) nanofiber mats, respectively. Between 30 and 180 min, the cumulative amount of CA-liquid released from the chitosan/CA(5.0%)/PEO nanofiber mats is statistically higher than the CA-liquid released from the chitosan/CA(0.5%)PEO nanofiber mats. Overall, 545% more CA was released from the mats spun with a higher CA concentration. Without wishing to be bound by theory, the overall higher release of CA-liquid from the chitosan/CA(5.0%)/PEO nanofiber mats supports the physical entrapment of a proportion of the “unreacted” CA during electrospinning. At longer times (90 and 180 min), lower rates of release were observed, which could indicate a lower diffusion driving force.^{42,43}

While UV-Vis confirmed the release of CA-liquid from the chitosan-CA nanofiber mats, it is highly likely that a significant amount of CA will also occupy the surrounding air space due to the volatility of the essential oil.⁴⁴ This volatility emphasizes the importance of characterizing the vapor phase even though most previous studies only quantify release into the liquid phase.²⁷ GC was employed to quantify the CA-vapor released into the vial headspace (FIG. 4B). In contrast to the UV-Vis, for accurate GC data, larger vials (20 mL) were used for accurate GC data to gain more air space to increase the sampling volume. From samples acquired after 30 min it was determined that 0.6±0.1 μg of CA-vapor was released from the chitosan/CA(0.5%)/PEO nanofiber mats and that a statistically equivalent amount of CA-vapor, 0.9±0.3 μg, was released from the chitosan/CA(5.0%)/PEO nanofiber mats. At 90 min, the CA-vaporreleased from chitosan/CA(0.5%)/PEO (0.6±0.1 μg) and from the chitosan/CA(5.0%)/PEO (0.7±0.2 μg) nanofiber mats were statistically equivalent. After 180 min, the quantity of CA-vapor released from the chitosan/CA(5.0%)/PEO nanofiber mats was statistically higher than the quantity released from the chitosan/CA(0.5%)/PEO mats, 0.7±0.1 μg versus 0.3±0.1 μg. Statistically, the same quantity of CA occupies the vapor headspace at all three time points investigated for each chitosan/CA/PEO nanofiber mats system (0.5 and 5.0%) thus indicating that the CA-vapor releases quickly. The chitosan/CA(5.0%)/PEO nanofiber mats released 279% more CA-vapor after 180 min than chitosan/CA(0.5%)/PEO nanofiber mats. The use of different collection setups for liquid and vapor release means that we cannot plot a reliable total release curve. However, we can state that more vapor-CA is released than liquid-CA, which further emphasizes the importance of characterizing the vapor phase release even though most previous studies only quantify release into the liquid phase.

Literature reports three potential mechanisms for release: swelling-controlled, diffusion-controlled, and reaction-controlled, all of which are highly pH dependent.^{42,43} Without wishing to be bound by theory, the governing mechanism of release likely depends on whether the CA is attached or unattached to the chitosan. Solid-state ¹³C NMR informs that the majority of CA in our chitosan/CA(0.5 and 5.0%)/PEO nanofiber mats is physically incorporated and therefore swelling controlled. Unreacted CA is likely to be released by the swelling of the chitosan and PEO once the nanofiber mats are submerged in the isotonic solution. Initial release testing at a range of pHs indicated that minimal release of CA occurred in basic conditions (pH of 8 or greater) where the nanofibers would have minimal swelling (not shown). Based on previous literature, we hypothesize that the CA diffuses through the mixed polymer network following Fickian diffusion.⁴⁵

Antibacterial Activity of Chitosan/CA/PEO Nanofiber Mats Against E. coli.

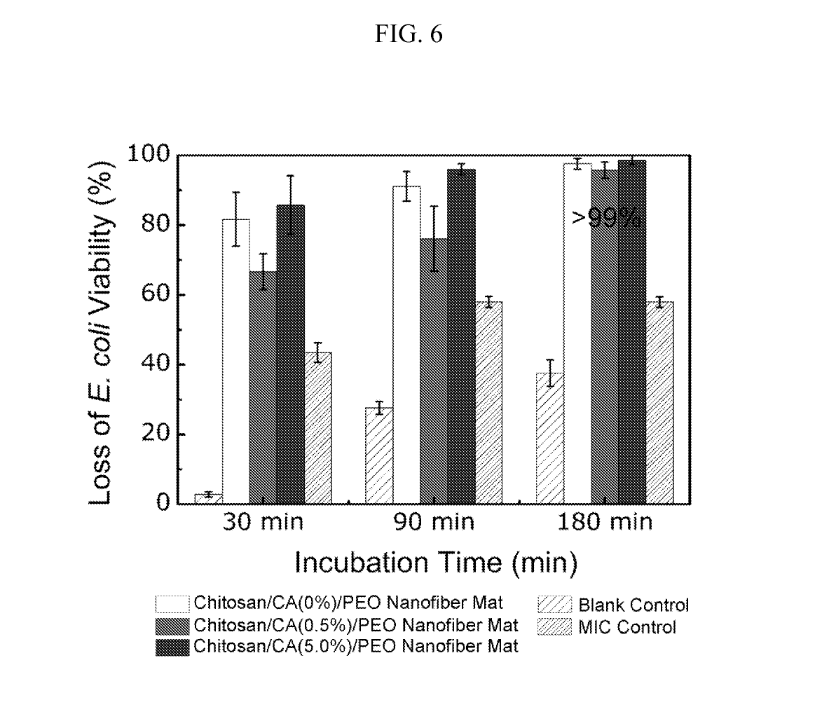

After only 30 min of nanofiber-bacteria contact, a high level of inactivation against E. coli is achieved (FIG. 6). Statistically, all three of our nanofiber mats demonstrated a complete inactivation at all incubation times evaluated (30, 90, and 180 min). Observed inactivation values for mats incubated for 180 min were at the limit of the accuracy or range of the bacterial viability assay. As displayed on FIG. 5, any value greater than 99% must be considered only as >99%.³³ These results are consistent with previous research²⁶, where nanofiber mats electrospun from a 5.0 wt % solution of chitosan/PEO (1/1) exhibited increased cytotoxicity against E. coli for 4 h. A low, statistically smaller loss of viability occurred in the blank control experiment (no nanofiber mat or antibacterial agent present).

The MIC control (3 mM CA solution, no nanofiber mat) allows for a comparison of the effect of CA has on E. coli inactivation.¹⁹ The MIC control statistically inactivated more E. coli than the blank control confirming previous reports that CA displays antibacterial activity against E. coli. Interestingly, after 30 and 180 min, the MIC control displayed a statistically lower level of antibacterial activity than chitosan/CA(0, 0.5, and 5.0%)/PEO nanofiber mats. At 90 min, the MIC control was statistically equivalent to the chitosan/CA(0.5%)/PEO nanofiber mats. These results suggest that at shorter times, CA alone may be less effective than the nanofiber mats.

The chitosan in the chitosan-CA nanofiber mats (0% CA) offer positively charged free amine groups, which can interact with the negatively charged cell surface of the bacteria. Chitosan/CA (0.5% and 5.0% CA)/PEO nanofiber mats offer an additional mechanism to inactivate bacteria: direct contact between microbes and the CA. While nanofiber mats containing attached CA offer less free amines to interact with miocrobes, the statistical equivalence of antibacterial efficacy achieved by all three chitosan/CA/PEO nanofiber systems confirms that by a using a combination of chitosan and CA killing mechanisms, a complete inactivation of E. coli is achieved.

Antibacterial Activity of Chitosan/CA/PEO Nanofiber Mats Against P. aeruginosa.

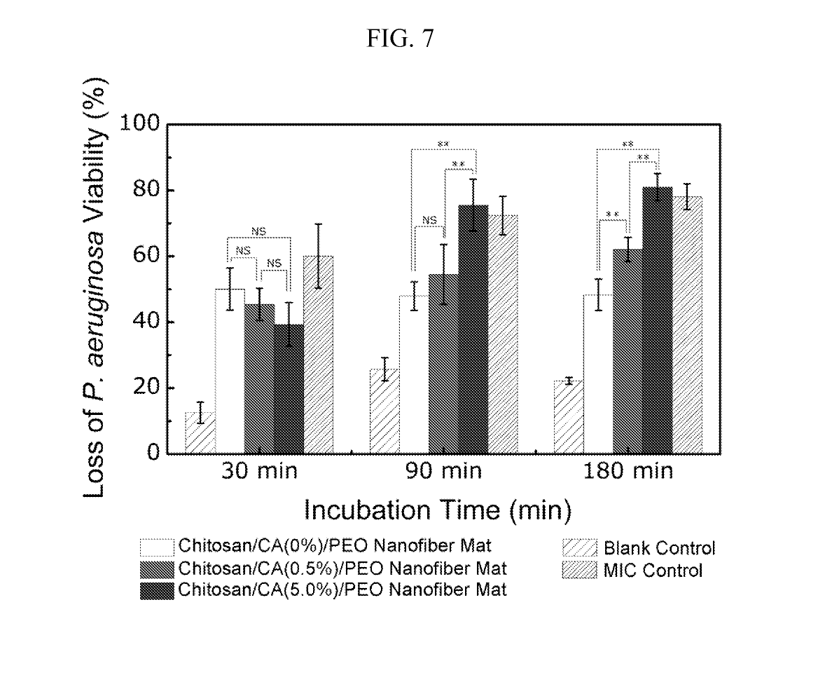

The efficacy of any chitosan-based nanofiber mats against P. aeruginosa has not yet been reported. Notably, fewer antibiotics are effective against this strain of microbe. FIG. 7 displays that after 30 min, statistically equal levels of P. aeruginosa inactivation were demonstrated by all three nanofiber mats. An initial efficacy of 50±6% against P. aeruginosa was demonstrated by the chitosan/CA(0%)/PEO nanofiber mats confirming the strong intrinsic cytotoxicity of chitosan, which was statistically equivalent to the MIC control. Once 90 min of incubation was reached, the chitosan/CA(5.0%)/PEO nanofiber mats achieved a higher rate of inactivation (76±8%) compared to the chitosan/CA(0 and 0.5%)/PEO nanofiber mats (48±4% and 54±9%). The higher antibacterial activity of chitosan/CA(5.0%)/PEO nanofiber mats is most likely due to the higher release of CA. The MIC control at 90 min inactivated P. aeruginosa at a statistically equivalent level as the chitosan/CA(0.5 and 5.0%)/PEO nanofiber mats, thus validating the contribution of CA to the increased antibacterial activity exhibited by the chitosan/CA(5.0%)/PEO nanofiber mats. After 180 min, chitosan/CA(0, 0.5, and 5.0%)/PEO nanofiber mats.⁵²

### Example 2

### Materials and Chemicals

Low weight chitosan (LMW CS, poly(D-glucosamine), MW=460,000 Da), medium molecular weight chitosan (MMW CS, poly(D-glucosamine), MW=1,000,000 Da) and poly(ethylene oxide) (PEO, Mw=600,000 Da) were used as received from Sigma Aldrich (St. Louis, Mo.). A modified medium molecular weight chitosan (MMW-MOD CS, MW=1,000,000 Da) was synthesized to provide a direct comparison of molecular weight and the degree of deacetylation. MMW-MOD CS was fabricated through the deacetylation of the MMW CS by suspending 5 g of MMW CS in 100 mL of 45% w/w NaOH. The solution was heated at 70° C. for 45 min. The MMW CS was then filtered and washed with deionized water until a neutral pH was achieved. The resultant powder was then dried for 12 hr in a vacuum oven at 25° C. Cinnamaldehyde (CA, ≧93%, FG, Mw=132.16 Da) and hydrocinnamic alcohol (H-CA, ≧98%, FCC, MW=136.19 Da) were investigated.

Stability.

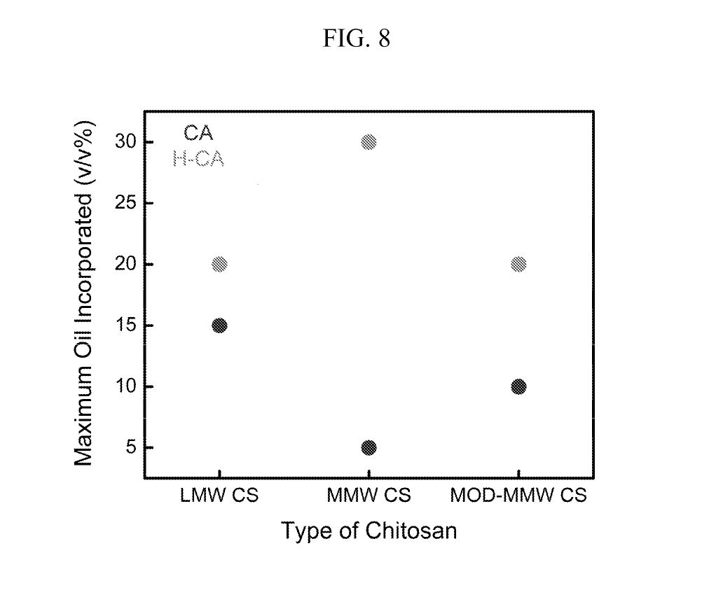

The stability of each oil loaded in LMW, MMW, and MMW-MOD CS:PEO solutions was performed at a total polymer concentration of 2 w/v %. All experiments utilized the polymer solution containing equal ratio of chitosan and PEO in a 0.5 M aqueous acetic acid solution. CA or H-CA was added at 0.5, 1.0, 1.5, 3.0, 5.0, 7.0, 10, 15, 20, and 30 v/v (organic/aqueous) % to the dissolved polymer solutions and mixed for 24 hr. The stability of each suspension was quantified by (1) maximum incorporation of the oil after mixing, as well as the presence of phase splitting (2) after 24 hr and (3) after 7 days. The maximum incorporation is displayed in FIG. 8. A higher molecular weight paired with a high DA % resulted in a lower incorporation of CA and a higher incorporation of H-CA. After 24 hrs, solutions containing either H-CA or CA phase split above 1.5%. After 7 days, all solutions phase separated.

Viscosity.

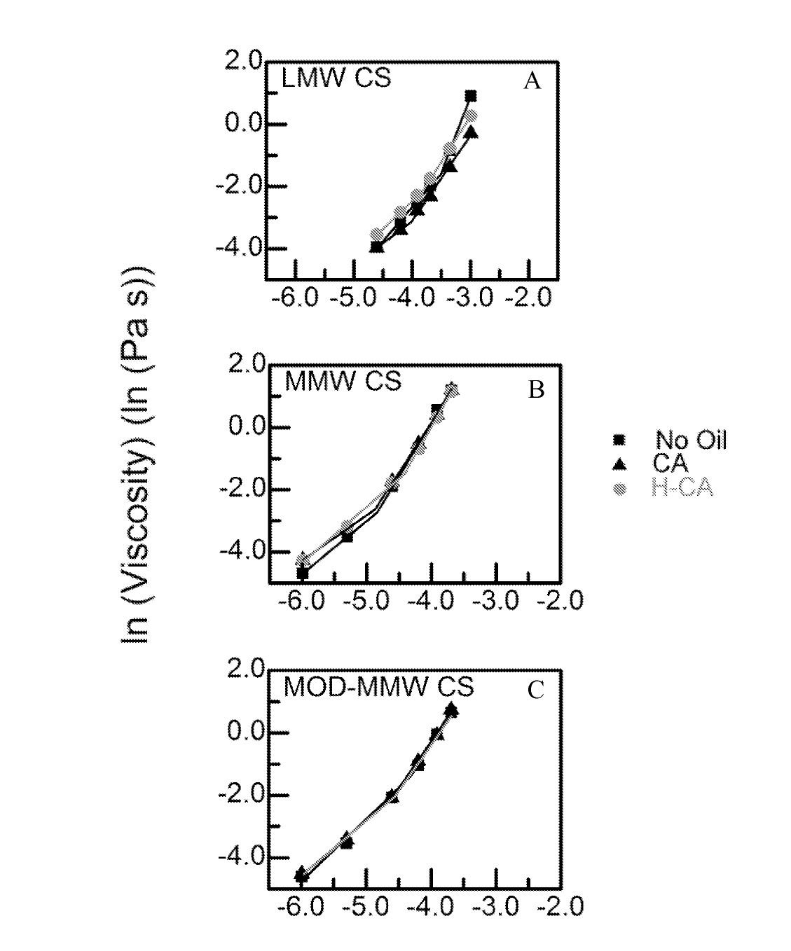

Viscosity measurements were performed in a Kinexus Pro rheometer using a concentric cylinder geometry, with a diameter of 25 mm, horizontal gap of 1 mm, and run using a vertical gap of 1 mm. A viscosity stress sweep was conducted from 0.1 to 10 Pa. A Newtonian plateau was observed within this range, and the average value of the plateau was reported. Measurements were conducted at 25° C. There were no signs of phase separation over the course of measurement. The addition of CA or H-CA did not result in a change in the rheological measurements indicating that the same minimal polymer concentration is needed to electrospin solutions containing CA or H-CA (FIGS. 9A-C).

Electrospinning and Resultant Fiber Morphology.

LMW, MMW, and MMW-MOD CS:PEO (1:1 weight ratio) solutions were electrospun at total polymer concentrations ranging from 1 to 5.0 w/v % to determine the necessary polymer concentration for entanglement. To electrospin these solutions a constant feed rate of 60 μL/min, a separation distance of 120 mm and an applied voltage of 35 kV was utilized. For MMW or MMW-MOD CS:PEO solutions, the total polymer concentration needed to form fibers was 2.0-2.5%. Solutions higher than 2.5% polymer became challenging to electrospin due to their high viscosity. Solutions containing LMW CS:PEO electrospun at 3.5-5.0% total polymer concentration and solutions higher than 5.0% were too viscous to electrospin.

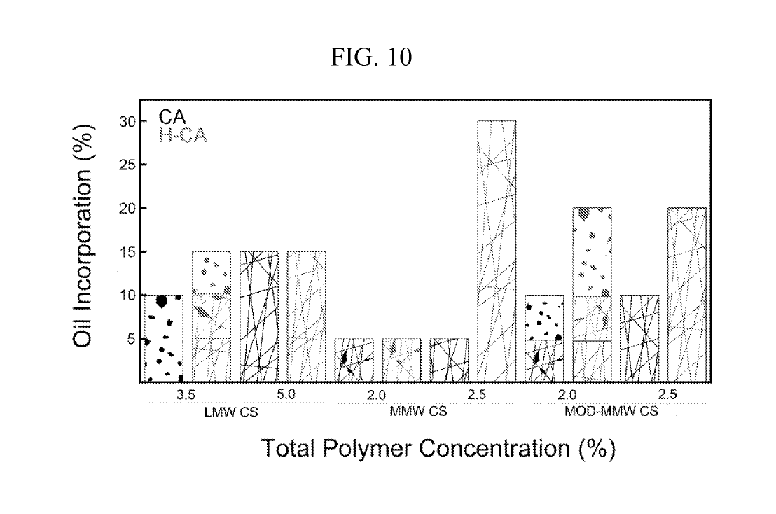

The quantity of oil (H-CA and CA) added to LMW, MMW, and MMW-MOD CS:PEO solutions was based on the initial stability test of each oil in each specific polymer solution and the total polymer concentration needed to form fibers. Once CA or H-CA was fully incorporated into the solution, it was electrospun at a constant feed rate of 60 μL/min, a separation distance of 120 mm and an applied voltage of 35 kV. The morphology produced by each type of solution electrospun is compiled in FIG. 10.

### CONCLUSION

Chitosan-CA derivatives were successfully prepared via a Schiff base reaction in aqueous acetic acid solutions maintained at a pH of 4. Solution and solid state NMR on precursor solutions and as-spun mats respectively determined that the attached-CA and unattached-CA were present in both. At physiological conditions, the polymer nanofiber mats released the CA-liquid and CA-vapor. Over 180 min, the quantity of CA-vapor was two orders of magnitude greater than CA-liquid with statistically higher levels of release from chitosan/CA(5.0%)/PEO nanofiber mats in both phases. Within 30 min, chitosan/CA(0%)/PEO nanofiber mats achieved a full inactivation of E. coli. Exploration into the cytotoxicity of the chitosan/CA(0%)/PEO nanofiber mats towards P. aeruginosa had not yet been demonstrated, and was determined to have a strong effect, inactivating ˜50% of the microbe. The release of CA from the chitosan/CA(0.5 and 5.0%)/PEO nanofiber mats directly influenced their cytotoxicity against P. aeruginosa. After 180 min, 81±4% of the P. aeruginosa, was inactivated by the chitosan/CA(5.0%)/PEO nanofiber mats. This work is the first demonstration that an essential oil can be incorporated into and successfully delivered from nanofiber mats.

### REFERENCES

(1) Donlan, R. M. Emerging infectious diseases 2002, 8, 881-90.
(2) Jr, F. R. In Pseudomonas aeruginosa, the Opportunist, Pathogensis and Disease; Fick R B Jr, Ed.; 1993.
(3) Page, M. G. P.; Heim, J. Curr. Opin. Pharmacol. 2009, 9, 558-65.
(4) Kleven, R.; Edwards, J.; Richards, C.; Horan, T. Public Health Reports 2007, 122, 160-166.
(5) Carmeli, Y.; Troillet, N.; Eliopoulos, G. M.; Samore, M. H. Antimicrob. Agents Chemother. 1999, 43, 1379-82.
(6) Hota, S.; Hirji, Z.; Stockton, K.; Lemieux, C.; Dedier, H.; Wolfaardt, G.; Gardam, M. A. Infect. Control Hosp. Epidemiol. 2009, 30, 25-33.
(7) Rasmussen, T. B.; Givskov, M. Med. Microbiol. 2006, 296, 149-61.
(8) De Kievit, T. R. Microbiol. 2009, 11, 279-88.
(9) Sauer, K.; Camper, A. K.; Ehrlich, G. D.; Costerton, J. W.; Davies, D. G. Bacteriol. 2002, 184, 1140-1154.
(10) Bakkali, F.; Averbeck, S.; Averbeck, D.; Idaomar, M. Food Chem. Toxicol. 2008, 46, 446-75.
(11) Pattnaik, S.; Suramanyam, V. R.; Bapaji, M.; Kole, C. R. Microbiol. 1997, 89, 39-46.
(12) Burt, S. Int. J. Food Microbiol. 2004, 94, 223-53.
(13) Hammer, K. A.; Carson, C. F.; Riley, T. V J. Appl. Microbiol. 1999, 86, 985-90.
(14) Kavanaugh, N. L.; Ribbeck, K. Appl. Environ. Microbiol. 2012, 78, 4057-61.
(15) Bouhdid, S.; Abrini, J.; Amensour, M.; Zhiri, A.; Espuny, M. J.; Manresa, A. J. Appl. Microbiol. 2010, 109, 1139-49.
(16) Niu, C.; Gilbert, E. S. Appl. Environ. Microbiol. 2004, 70, 6951-6.
(17) Niu, C.; Afre, S.; Gilbert, E. S. Lett. Appl. Microbiol. 2006, 43, 489-94.
(18) Jia, P.; Xue, Y. J.; Duan, X. J.; Shao, S. H. Lett. Appl. Microbiol. 2011, 53, 409-16.
(19) Zodrow, K. R.; Schiffman, J. D.; Elimelech, M. Langmuir 2012, 39, 13993-9.
(20) Ojagh, S. M.; Rezaei, M.; Razavi, S. H.; Hosseini, S. M. H. Food Chem. 2010, 122, 161-166.
(21) Engel, Y.; Schiffman, J. D.; Goddard, J. M.; Rotello, V. M. Materials Today 2012, 15, 478-485.
(22) Pine, S. H.; Hendrickson, J.; Cram, D. J.; Hammono, G. S. Organic Chemistry; 4th ed.; McGrawHill: New York, 1980; pp. 285-286.
(23) Cordes, E. H.; Jencks, W. P. J. Am. Chem. Soc. 1962, 84, 832-837.
(24) Rabea, E. I.; Badawy, M. E.-T.; Stevens, C. V; Smagghe, G.; Steurbaut, W. Biomacromolecules 2003, 4, 1457-65.
(25) Schiffman, J. D.; Schauer, C. L. Biomacromolecules 2007, 8, 2665-7.
(26) An, J.; Zhang, H.; Zhang, J.; Zhao, Y.; Yuan, X. Colloid Polym. Sci. 2009, 287, 1425-1434.
(27) Yang, J.; Kuang, X.; Li, B.; Zhou, B.; Li, J.; Cui, B.; MA, M. J. Food Safety 2012, 32, 189-197.
(28) Talrose, V.; Yermakov, A. N.; Usov, A. A.; Goncharova, A. A.; Leskin, A.; Messineva, N. A.; Trusova, N. V.; Efimkina, M. V. NIST Chemistry WebBook, NIST Standard Reference Database Number 69, Eds. P. J. Linstrom and W. G. Mallard, National Institute of Standards and Technology 2007.
(29) Friedman, M.; Kozukue, N.; Harden, L. A. J. Agric. Food Chem 2000, 48, 5702-5709.
(30) American Society for Testing and Materials: ASTM E2149-01 2004.
(31) Fu, J.; Ji, J.; Yuan, W.; Shen, J. Biomaterials 2005, 26, 6684-92.
(32) Chen, L.; Bromberg, L.; Hatton, T. A.; Rutledge, G. C. Polymer 2008, 49, 1266-1275.
(33) Schiffman, J. D.; Wang, Y.; Giannelis, E. P.; Elimelech, M. Langmuir 2011, 27, 13159-64.
(34) Hirai, A.; Odani, H.; Nakajima, A. Polymer Bulletin 1991, 26, 87-94.
(35) Varum, K. M.; Antohonsen, M. W.; Grasdalen, H.; Smidsrod, O. Carbohydrate Research 1991, 211, 17-23.
(36) Fernandez-Megia, E.; Novoa-Carballal, R.; Quiñoá, E.; Riguera, R. Carohydr. Polym. 2005, 61, 155-161.
(37) Guinesi, L. Carbohydr. Polym. 2006, 65, 557-561.
(38) Ganjian, I.; Baumgarten, R. L.; Valenzuela, R. J. J. Chem. Educ. 1992, 69, 511.
(39) No, H. K.; Park, N. Y.; Lee, S. H.; Meyers, S. P. Int. J Food Microbiol. 2002, 74, 65-72.
(40) Zalipsky, S. Adv. Drug Deliver. Rev. 1995, 16, 157-182.
(41) Pakravan, M.; Heuzey, M.-C.; Ajji, A. Polymer 2011, 52, 4813-4824.
(42) Peppas, N. A.; Bures, P.; Leobandung, W.; Ichikawa, H. Eur. J. Pharm. Biopharm. 2000, 50, 27-46.
(43) Siepmann, J.; Peppas, N. A. Int. J. Pharm 2011, 418, 6-12.
(44) Edris, A. E. Phytotherapy Research 2007, 21, 308-23.
(45) Verreck, G.; Chun, I.; Rosenblatt, J.; Dijck, A. V; J, M.; Noppe, M.; Brewster, M. E. J. Control. Release 2003, 92, 349-60.
(46) Helander, I. M.; Nurmiaho-Lassila, E. L.; Ahvenainen, R.; Rhoades, J.; Roller, S. Int. J. Food Microbiol. 2001, 71, 235-44.
(47) Liu, H.; Du, Y.; Wang, X.; Sun, L. Int. J. Food Microbiol. 2004, 95, 147-55.
(48) Oussalah, M.; Caillet, S.; Lacroix, M. J. Food Prot. 2006, 69, 1046-55.
(49) López, P.; Sanchez, C.; Batlle, R.; Nerín, C. J. Agric. Food Chem 2007, 55, 4348-56.
(50) Pakravan, M.; Heuzey, M.-C.; Ajji, A. Polymer, 2011, 52, 4813-4824.
(51) Jatunov, S., Franconetti, A., Gomez-Guillen, M., & Cabrera-Escribano, F., Sciforum Electronic Conference Series, 2012, 16.
(52) Rieger, K. A.; Schiffman, J. D. Carbo. Polym., 2014, 113, 561-568.

What is claimed is:

1. A method of preparing a nanofiber mat, the mat comprising an essential oil or a volatile organic thereof, the method comprising electrospinning a mixture of chitosan, poly(ethylene oxide), an acid, and an essential oil or a volatile organic thereof, wherein the mixture of has a pH of approximately 4, thereby preparing a nanofiber mat comprising an essential oil.

2. The method of claim 1, wherein the ratio of chitosan to poly(ethylene oxide) is approximately 1:1.

3. The method of claim 1, further comprising the step of mixing the chitosan and poly(ethylene oxide) for approximately 24 hours.

4. The method of claim 1, wherein the acid is acetic acid.

5. The method of claim 1, wherein the applied voltage for the electrospinning is approximately 25 kV.

6. The method of claim 1, wherein the molecular weight of the chitosan is between about 460,000 Da and about 1,000,000 Da.

7. The method of claim 1 wherein the essential oil is cinnamaldehyde.

8. The method of claim 7, wherein the amount of cinnamaldehyde is approximately 0.5 v/v %.

9. The method of claim 7, wherein the amount of cinnamaldehyde is approximately 5.0 v/v %.

10. The method of claim 1 wherein the essential oil or volatile organic thereof is hydrocinnamic alcohol.

11. A nanofiber mat prepared by the method of claim 1.

12. The nanofiber mat of claim 11, wherein the ratio of chitosan to poly(ethylene oxide) is approximately 1:1.

13. The nanofiber mat of claim 11, further comprising the step of mixing the chitosan and poly(ethylene oxide) for approximately 24 hours.

14. The nanofiber mat of claim 11, wherein the acid is acetic acid.

15. The nanofiber mat of claim 11, wherein the essential oil is cinnamaldehyde and the amount of cinnamaldehyde is approximately 0.5 v/v %.

16. The nanofiber mat of claim 11, wherein the essential oil is cinnamaldehyde and the amount of cinnamaldehyde is approximately 5.0 v/v %.

17. The nanofiber mat of claim 11, wherein the applied voltage for the electrospinning is approximately 25 kV.

18. The nanofiber mat of claim 11, wherein the nanofibers have a diameter of between approximately 38 nm and 55 nm.

19. A method of inactivating microbes comprising contacting a nanofiber mat prepared as in claim 1 with the microbe under time and conditions sufficient for the microbe to be partially, or completely, inactivated.

20. The method of claim 19 wherein the microbe is E. coli or P. aeruginosa.

21. The method of claim 19 wherein the bacteria has formed a biofilm and the time and conditions the nanofiber mat is contacted with the microbial biofilm is sufficient for the dispersion of the biofilm.
